(19) United States
(12) Patent Application Publication
(10) Pub. No.: US 2024/0173509 A1
(43) Pub. Date: May 30, 2024

(54) PROBE FOR GAS SUPPLY SYSTEM

(71) Applicant: Drägerwerk AG & Co. KGaA, Lübeck (DE)

(72) Inventors: Thomas ROSSEN, Lübeck (DE); Michael HEIDSCHMIDT, Lübeck (DE); Andreas BRANDT, Lübeck (DE); Vladimir SAVCHUK, Lübeck (DE); Jan DANNEMANN, Lübeck (DE)

(21) Appl. No.: 18/520,746

(22) Filed: Nov. 28, 2023

(30) Foreign Application Priority Data
Nov. 29, 2022 (DE) 10 2022 131 453.7

(51) Int. Cl. A61M 16/10; A61M 16/00

(52) U.S. Cl. CPC A61M 16/1005; A61M 16/0003; A61M 16/104

(57) ABSTRACT

A two-part probe (5) is for a pneumatic gas delivery system (100) with a breathing system (1) and a pneumatic interface (3) of a ventilator (700) or anesthesia device (500). The two-part probe (5) enables gas exchange (29) between the breathing system (1) and the pneumatic interface (3) by means of a channel arrangement (10).

### CROSS REFERENCE TO RELATED APPLICATIONS

This application claims the benefit of priority under 35 U.S.C. § 119 of German Application 10 2022 131 453.7, filed Nov. 29, 2022, the entire contents of which are incorporated herein by reference.

### TECHNICAL FIELD

The present invention pertains to configurations of probes for a gas delivery system. The configurations of probes may be used in a pneumatic gas delivery system having a breathing system and a pneumatic interface of a ventilator or anesthesia machine.

### BACKGROUND

Such probes are used in a wide range of applications in the field of industrial technology. Probes are used in particular when a physical condition is to be transferred from one side of a wall to the other side of the wall during process execution or process monitoring through transformations of containers and tanks. In addition, probes can be used to transport gas quantities as well as to feed gas quantities from one side of a wall to the other side of the wall. Pressure equalization between the two sides of the wall is also possible by means of probes.

A ventilator or anesthesia device has a breathing system (respiratory system) which is responsible for providing gas mixtures and/or gas quantities for supplying a patient with inhalation gas and for carrying away exhalation gas from the patient. For this purpose, breathing tubes, configured in the form of an inspiratory and an expiratory breathing tube, are connected to the breathing system for connection to the patient. In operation, the breathing system is connected to further components of the ventilator or anesthesia machine via a pneumatic interface. Further components are, for example, actuators, such as passive or actively controlled gas inlet valves, gas outlet valves or check valves, actively controlled dosing valves or dosing elements, as well as delivery devices for gas quantities, such as blower drives or piston drives. Other components include sensors, such as pressure sensors, temperature sensors, flow rate sensors, gas sensors, humidity sensors.

JP2012024620 A discloses in simple form structured and highly reliable connection of an interconnection line with a system for hemodialysis. US2013158521 A1 discloses a two-part connection system having a threaded connection for selectively supplying fluid to a device from one of a plurality of fluid sources having a first inlet for connecting a first fluid source and a second inlet for connecting a second fluid source.

EP2586487 A1 discloses a two-part connection system for a transport of medical fluids. U.S. Pat. No. 9,795,757 B2 discloses a fluid inlet adapter for a fluidic connection. The adapter provides a fluid inlet that can be configured to provide only one of two possible fluids and a machine-readable indication of which fluid is being accepted. US2007076401 A1 discloses a threaded connector for a fluidic connection.

A pneumatic connection can be made from the breathing system to the other components of the ventilator or the anesthesia device via connecting elements configured as probes arranged in the pneumatic interface. Both the ventilation tubes and the breathing system come into contact with exhaled gas from the patient and must therefore be hygienically processed (processed/reprocessed) after completion of the therapeutic measures on the patient before being reused on a next patient. The breathing system can be separated from the ventilator or anesthesia machine and removed from the ventilator or anesthesia machine in order to perform the hygienic processing. After the breathing system has been hygienically processed, it is advisable for hygienic reasons, before the breathing system is repositioned in the ventilator or anesthesia machine and before the ventilator or anesthesia machine is put back into operation, to also design the probes arranged in or at the pneumatic interface in such a way that the probes can also be included in a hygienic processing concept.

### SUMMARY

It is an object of the present invention to provide configurations of probes for an anesthesia or respiratory machine which enable the probes to be included in an improved hygienic concept for the operation of the anesthesia or respiratory machine.

The task is solved by a probe with features according to this disclosure.

Advantageous embodiments of the invention are presented in this disclosure and are explained in more detail in the following description with partial reference to the figures.

According to the invention, the probe is configured or constructed in at least two parts. The at least two-part configuration or constructed probe is comprised of a probe element and a conduit element, which can be connected to each other.

According to the invention, at least two-part configured or formed probes in a pneumatic gas delivery system enable gas exchange between the breathing system by means of the pneumatic interface with other components of the ventilator or anesthesia machine.

On the one hand, the probes according to the invention, which are configured or constructed in at least two parts, can enable transmission of pneumatic and/or physical situations (states) which are present in the breathing system to sensors or actuators in the respiratory or anesthesia device via the pneumatic interface by means of the enabled gas exchange between the breathing system and other components of the respiratory device or anesthesia device. Pneumatic situations are, for example, an absolute pressure or relative pressure given or prevailing in the breathing system. Physical situations are, for example, temperatures, gas concentrations, relative or absolute humidities, flow rates, pressure differences as a measure of flow rates.

On the other hand, the probes according to the invention, which are configured or constructed in at least two parts, can also enable gas quantities to be fed into the breathing system by means of the gas exchange enabled between the breathing system and other components of the ventilator or anesthesia device. A supply of gas quantities into the breathing system can include, for example, a metering or dosing of gas quantities of medical air (air), oxygen (O₂), other gases (NO, helium, HeliOx=oxygen-helium mixture), anesthetic gases (halothane, enflurane, sevoflurane, desflurane, isoflurane), nitrous oxide (N₂O).

In addition, the probes according to the invention, which are configured or constructed in at least two parts, can also enable a continuation of gas quantities from the breathing system by means of the enabled gas exchange between the breathing system with other components of the ventilator or anesthesia device. A continuation of gas quantities from the breathing system may include, for example, a continuation of exhaled gases into the circuit system of an anesthesia machine, into an anesthetic gas or anesthetic gas delivery system (NGF, AGS).

According to the invention, the probe for a pneumatic gas delivery system comprising a breathing system and a pneumatic interface of a ventilator or anesthesia machine is formed in the following manner described below.

The probe is formed at least in two parts and is comprised of a combination of a probe element with a conduit (feedthrough) element. The two-part configuration can also be described or referred to as a configuration of a probe with an upper part (probe element) and a lower part (conduit element). The probe element is connectable to the conduit element. The probe element and the conduit element, when connected to each other, together form the probe. Such probes are often also referred to as grommets or fittings. The probe is configured by means of the combination of a probe element with a conduit element and by means of at least one channel arrangement internal to the probe, to provide a pneumatic or fluidic conductive connection between the breathing system and the pneumatic interface. The channel arrangement is formed with a probe channel and feedthrough channel. By means of the channel arrangement, gas exchange is enabled between the probe element and the conduit element.

The probe can be connected to the pneumatic interface or components of the ventilator or anesthesia machine by means of at least one connector element arranged on the conduit element. Connection elements—often also called connectors—enable the connection of one end of hose lines or tubes, which can then be connected to the other end with components, for example with a sensor interface or with sensors (pressure sensors, temperature sensors, gas sensors, humidity sensors, flow rate sensors), actuators (passive or active valves), ventilation drives (blower drives, blower drives, piston drives), gas inputs, or gas outputs of the anesthesia or ventilation device.

In a preferred embodiment, the probe, configured as and functioning as a measuring probe, enables a transmission of a pneumatic situation (absolute pressure, relative pressure) and/or physical situation (temperatures, gas concentrations, relative or absolute humidities, flow rates, pressure differences as a measure for flow rates) to the breathing system via the enabled gas exchange by means of the pneumatic interface by means of at least one probe opening arranged in the probe element and by means of at least one first probe channel located inside the probe element and by means of at least one first feedthrough channel located inside the conduit element, a transmission of a pneumatic situation (absolute pressure, relative pressure) and/or physical situation (temperatures, gas concentrations, relative or absolute humidities, flow rates, pressure differences as a measure of flow rates) in the breathing system to a sensor interface and/or sensors (pressure sensors, temperature sensors, gas sensors, humidity sensors, flow rate sensors) connected to a pneumatic interface.

The probe opening arranged in the probe element, together with the upper part of the probe element, which may also be referred to as the probe head, is formed in a substantially rounded shape, preferably tapered. The taper enables a flow around the probe head in the breathing system. In addition, sharp edges are avoided in this way, which helps to prevent damage to sealing elements and also enables a force-saving joining of the interface with the probe to the breathing system during assembly by the user. According to the invention, the first probe channel internal to the probe element and the first feedthrough channel internal to the conduit element form a continuous channel arrangement in the probe, which enables and provides the transmission of pneumatic and/or physical situation to the sensor interface. The pneumatic and/or physical situation can be provided in this way to, to the sensor interface and/or to sensors (pressure sensors, temperature sensors, gas sensors, humidity sensors, flow rate sensors). This type of two-part measuring probe makes it possible, in particular, to provide a pressure level in the breathing system to a pressure sensor that is located outside the breathing system in an area of the anesthesia device or ventilator device that does not have to be processed regularly during hygienic processing. Thus, the two-part measuring probe represents a type of hygienic interface between the breathing system—which is configured to be hygienically processed—and other areas and components of the anesthesia device or ventilator device.

In a further preferred embodiment, the probe configured as and functioning as a gas inlet enables a supply of gas quantities from the pneumatic interface into the breathing system via the enabled gas exchange by means of the pneumatic interface by means of at least one probe opening arranged in the probe element and by means of at least one first probe channel located inside the probe element and by means of at least one first feedthrough channel located inside the conduit element. This method of a two-part probe as gas outlet represents a type of hygienic interface between the breathing system—which is configured to be hygienically processable—and other areas and components of the anesthesia device or ventilator device, which usually do not require hygienic processing or cleaning in regular clinical routine.

In a further preferred embodiment, the probe configured as and functioning as a gas outlet enables a continuation of gas quantities from the breathing system by means of the pneumatic interface via the enabled gas exchange by means of at least one probe opening arranged in the probe element and by means of at least one first probe channel located inside the probe element and by means of at least one first feedthrough channel located inside the conduit element. This way of a two-part configured probe as gas inlet represents a kind of a hygienic interface between the breathing system—which is configured to be hygienically processable—and other areas and components of the anesthesia device or ventilator, which usually do not require hygienic processing or cleaning in the regular clinical routine.

In a further preferred embodiment, the fluidically conductive connection made possible between the probe element and the conduit element can be configured as a detachable screw connection as a threaded combination with an externally threaded element and a corresponding internally threaded element. A threaded combination with an externally threaded element and an internally threaded element corresponding thereto enables a simple joining as well as separation of the probe element and the conduit element. In a preferred embodiment, the probe element can be formed with an externally threaded element and the conduit element can be formed as an internally threaded element.

In an alternative preferred embodiment, the conduit element may be formed with an externally threaded member and the probe element may be formed as an internally threaded member.

In a further preferred embodiment, the fluidically conductive connection between the probe element and the conduit element can be configured as a screw connection between the probe element and the conduit element that can be released in a tool-free manner. A screw connection between the probe element and the conduit element that can be released in a tool-free manner results in the advantage that the personnel can separate the probe element and the conduit element during hygienic processing without special tools and thus remove the probe element and refit it with the probe element during hygienic processing without great effort.

In a further preferred embodiment, the probe element may comprise at least one second probe channel and the conduit element may comprise at least one second feedthrough channel. An embodiment with at least one second probe channel and with at least one second feedthrough channel forms a two-channel channel arrangement within the probe, which can be used to measure two physical and/or pneumatic measurands in the breathing system separately and/or independently of each other and can thus form a combination of two measuring probes. Such an embodiment can also form a combination of a measuring probe with a gas input. Such an embodiment may likewise form a combination of a measurement probe with a gas output. Such an embodiment may also form a combination of a gas input with a gas output. Such an embodiment may also form a combination of a gas inlet with another gas inlet. Such an embodiment can also form a combination of a gas outlet with a further gas outlet.

In a further preferred embodiment, the probe element may be formed by means of a further probe opening, a pneumatic or fluidic conductive connection between the breathing system and the pneumatic interface may be provided via the second probe channel and the second feedthrough channel, wherein the second probe channel and the first feedthrough channel form a coaxial arrangement with each other, wherein one of the at least two probe channels is internally disposed within the other probe channel, and wherein the second feedthrough channel and the first feedthrough channel form a coaxial arrangement with each other as a dual channel arrangement wherein one of the at least two feedthrough channels is internally disposed within the other feedthrough channel. A two-channel coaxial two-part channel arrangement can be formed within the probe in this manner. This two-channel coaxial two-part channel arrangement can easily allow joining and separation of the probe element and the conduit element in the form of a screw connection - even without tools. A coaxially formed two-part channel arrangement can thus form a combination of two measuring probes. Such a coaxially formed two-part channel arrangement can also form a combination of a measuring probe with a gas inlet. Such a coaxially formed two-part channel arrangement can also form a combination of a measuring probe with a gas outlet. A coaxial two-part channel arrangement can also form a combination of a gas inlet with a gas outlet.

Such a coaxial two-part channel arrangement can also form a combination of a gas inlet with a further gas inlet. Such a coaxial two-part channel arrangement can also form a combination of a gas outlet with a further gas outlet.

In a further preferred embodiment, a volume element (buffer volume element) can be arranged or integrated in or on the probe, the probe element, the conduit element, the channel arrangement, one of the probe channels, one of the feedthrough channels or one of the connection elements, the volume element being configured to buffer pressure differences, reduce pressure surges in the breathing system or the pneumatic interface or reduce movements of gas quantities between the breathing system and the pneumatic interface. In a preferred manner, the volume element is dimensioned in such a way that, during operation, temporarily given pressure changes, in particular pressure increases or pulsating flow processes due to pressure fluctuations in the breathing system, can be buffered in the volume of the volume element in internal flow-carrying components of the anesthesia or ventilation device. An overview of the pneumatic situation in and around the breathing system can be found in the diagram in FIG. 8. The physical quantities essential for dimensioning the volume element are listed below:
p_ breathing system: pressure in the breathing system;
V_probe: inner, flow-carrying volume of the probe;
V_hose: inner, flow-carrying volume of the hoses;
V_Filter: inner, flow-carrying volume of the filter;
V_div: other flow-carrying volumes (other components, dead spaces pressure sensors, etc.);
V_insides: internal flow-conducting components of the anesthesia or ventilation device;
dV_compression: Volume change of the internal gas quantity due to compression during operation.

In an exemplary embodiment to illustrate the dimensioning of the volume element, a connection element of the probe (V_probe) is connected to a pressure sensor by means of hose lines (V_hose) by means of a filter (V_filter) arranged in series. A valve is connected to the probe (V_probe) via the connection element or another connection element of the probe by means of hose lines (V_hose) and possibly other components (V_div). To ensure that no contamination can occur between the breathing system and internal flow-carrying components as a result of compression of the internal flow-carrying gas volume, the following condition (formula 1) must apply:

$$d {V}_{C o m p r e s s i o n} = ( 1 - \frac{{p}_{BreathingSystem , min}}{\begin{matrix}{p}_{BreathingSystem , min} + \\ Δ {p}_{BreathingSystem , max}\end{matrix}} ) {V}_{I n s i d e} < {V}_{P r o b e} \qquad Formula 1$$

Using formula 2 and with further assumptions, formula 3 yields a factor k as a dimensioning aid between compressed volume (dV_compression) and the volume of the internal flow-conducting components (V_insides) of the anesthesia or ventilation device.

V_{inside}=V_{probe}+V_{Filter}+V_{Hose}+V_{div} Formula 2

The following assumptions apply to the ambient conditions in the breathing system:
P_{BreathingSystem,min} is the minimum pressure in the breathing system. Typically, this is the ambient pressure at the maximum permissible operating altitude, e.g., an ambient pressure at approx. 2000-3500 m altitude of 600 mbar to 710 mbar,
Δp_{BreathingSystem,max} is the maximum pressure stroke in the breathing system. Typically the maximum permissible inspiratory pressure (70-80 mbar).

The following formula 3 provides the factor k as a dimensioning aid.

$$( 1 - \frac{{p}_{BreathingSystem , min}}{{p}_{BreathingSystem , min} + Δ {p}_{BreathingSystem , max}} ) = k \qquad Formula 3$$

With the above assumptions, this results in a value for the factor k in a range of approximately k=0.105 to 0.135. If the internal volume of the flow-conducting components is, for example, 1,000.00 mm³, a suitable dimensioning for the volume of the two-part probe is a volume (V_probe) with a total size in a range of approximately 1,200.00 mm³ to 1,300.00 mm³. In principle, it does not matter how the volume is distributed between the probe element and the conduit element. The probe channel and the feed-through channel together form the desired probe volume (V_probe) of approximately 1,200.00 mm³.

In a further preferred embodiment, the probe element and/or conduit element may have at least one sealing element that provides a gas-tight connection to the conduit element and wherein conduit element has at least one sealing element that provides a gas-tight connection to the pneumatic interface. The gas-tight seal using sealing elements, for example in the form of sealing rings or O-rings, between the probe element and the conduit element ensures that the connection between the breathing system and the pneumatic interface is permanently configured without possible leaks during operation of the anesthesia device or ventilator device.

In a further preferred embodiment, the probe and/or the conduit element and/or the probe element and/or at least one of the sealing elements or sealing elements can be made of a plastic material, in particular an elastomer, and/or the probe and/or the conduit element the probe element can be made of a metallic material. Suitable metals are, for example, stainless steel alloys (stainless steel), brass, brass alloys, brass bronze, bronze, copper, aluminum, metals with passivated or coated chromium-plated surfaces. Suitable plastics are for example polyamide, polyimide, polyethylene, polysulfone, polyetheretherketone.

Suitable elastomers are, for example, rubbers such as ethylene-propylene-diene rubber (EPDM) or nitro-butadiene rubber (NBR), silicone rubber, silicone.

In a further preferred embodiment, an assembly may be formed with at least one probe, a breathing system, and a pneumatic interface.

At least one of the probes can be arranged on the breathing system in such a way that, in a configuration of the probe as a measuring probe, it is possible to record a pneumatic and/or physical situation of expiratory or inspiratory gas quantities.

At least one of the probes can be arranged on the breathing system in such a way that, in one embodiment of the probe as a gas inlet, it is possible to supply, in particular meter, quantities of gas into the breathing system via the pneumatic interface. In this way, gas quantities of fresh gas, for example as a gas mixture of medical air (Air), oxygen (O₂), possibly nitrous oxide (N₂O) and a volatile anesthetic agent (halothane, enflurane, sevoflurane, desflurane, isoflurane), provided by means of an anesthetic evaporator (Vapor), can be used by means of metering valves, are provided and introduced into the breathing system of an anesthesia machine, which can then be made available from the breathing system to the patient for inhalation as fresh inspiration gas. Probes may be used in an anesthesia machine, wherein amounts of gas exhaled by the patient and processed by a carbon dioxide absorber are added to the fresh gas, thereby ensuring that carbon dioxide (CO₂) is removed from the exhaled gases, but with exhaled amounts of volatile anesthetic gases returned back in the circuit for carrying out the anesthesia. In a breathing system of a ventilator, for example, gas quantities of medical air (Air), oxygen (O₂) and possibly other gases (NO, helium, HeliOx=oxygen-helium mixture) can be metered into the breathing system using metering valves.

The breathing system of the ventilator then provides this amount of gas mixture to the patient for inhalation via the pneumatic interface as fresh inspiratory gas.

At least one of the probes can be arranged on the breathing system of an anesthesia machine in such a way that the probe can be configured as a gas outlet to allow gas quantities to be conveyed from the breathing system of an anesthesia machine. In this way, for example, gas quantities of exhaled gas—controlled by means of an expiration valve (PEEP valve)—can be conducted from the breathing system via the pneumatic interface to the carbon dioxide absorber of the anesthesia device. The carbon dioxide absorber of the anesthesia machine can then remove the amounts of carbon dioxide (CO₂) exhaled by the patient from the exhaled gas. The processed gas can then be provided to the patient again as inspiratory gas for inhalation—as previously explained for the provision of inspiratory gas of an anesthetic gas by means of probes. At least one of the probes can be arranged on the breathing system of a ventilator in such a way that, in a configuration of the probe as a gas outlet, it enables gas quantities to be conveyed from the breathing system of a ventilator to the environment in a controlled manner by means of an expiration valve (PEEP valve).

In a further preferred embodiment, a pneumatic gas delivery system or anesthesia machine or ventilator may be formed with an arrangement.

Such an arrangement can use various sensors for this purpose, such as
at least one pressure sensor, and/or
at least one temperature sensor, and/or
at least one gas sensor, and/or
at least one humidity sensor, and/or
at least one flow rate sensor.

Such sensors can, by means of the pneumatic interface and the at least one probe to the connecting element or connecting lines connectable to the connecting elements, measure the pneumatic and/or physical situations (states) given in the breathing system and provide them to a control unit of the anesthesia machine or ventilator for controlling the operation of the anesthesia machine or ventilator.

For this purpose, the arrangement may include at least one connecting line between
the pressure sensor and/or
the temperature sensor and/or
the gas sensor and/or humidity sensor and/or
the flow rate sensor and
the at least one connection element at conduit element of the probe.

In a further preferred embodiment, the assembly or pneumatic gas delivery system or anesthesia machine or ventilator may include pneumatic or fluidic connections between the breathing system and the pneumatic interface by means of the at least two probes. These can be formed by mutually unique and unmistakable configurations of the thread combination on the probe element and/or unique and unmistakable configurations of the connection elements on the conduit element. Unmistakable configurations of the thread combination can be configured in such a way that the number of threads, the thread diameter, the thread length, the thread pitch of the probe element and conduit element are coordinated with each other, so that in the case of configurations with several probes at the pneumatic interface of an anesthesia device or ventilator device, it is ensured that in each case only the probe element intended for combination with conduit element can be screwed into conduit element and connected. Such a coordinated configuration of the at least two-part probes with probe elements and lead-through elements can ensure that possible mix-ups can be effectively prevented when the probe elements are replaced during hygienic processing.

By means of the following descriptions, with partial reference to the figures, aspects of the invention are explained in more detail. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a probe, a breathing system and a pneumatic interface;

FIG. 2 is perspective views showing a two-part probe with a probe element and a feed-through element;

FIG. 3 is a sectional view showing a two-part probe;

FIG. 4 is a sectional view showing a variant of a two-part probe;

FIG. 5 is a sectional view showing a variant of a two-lumen, two-part probe,

FIG. 6 is a sectional view showing a dimensioned representation of a probe element;

FIG. 7 is a sectional view showing a two-part and rotatable conduit element; and

FIG. 8 is a schematic view of a pneumatic situation in and on the breathing system.

### DESCRIPTION OF PREFERRED EMBODIMENTS

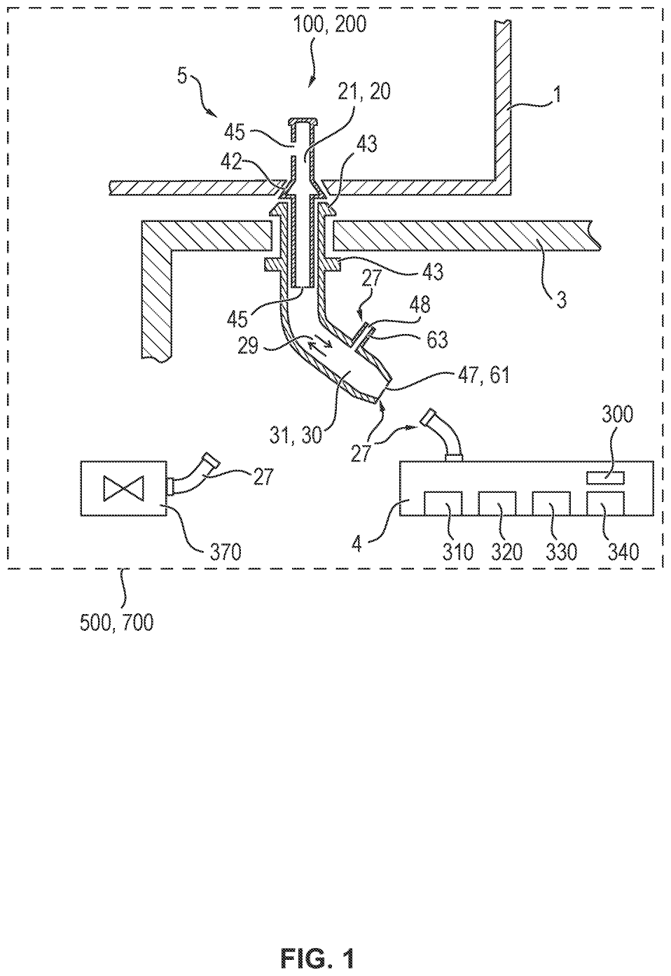

Referring to the drawings, FIG. 1 schematically shows an illustration of a two-part probe 5, a breathing system 1 and a pneumatic interface 3. The breathing system 1, the pneumatic interface 3 and the probe 5 together form a pneumatic gas delivery system 200 for an anesthesia machine 500 or a ventilator 700 as an assembly 100.

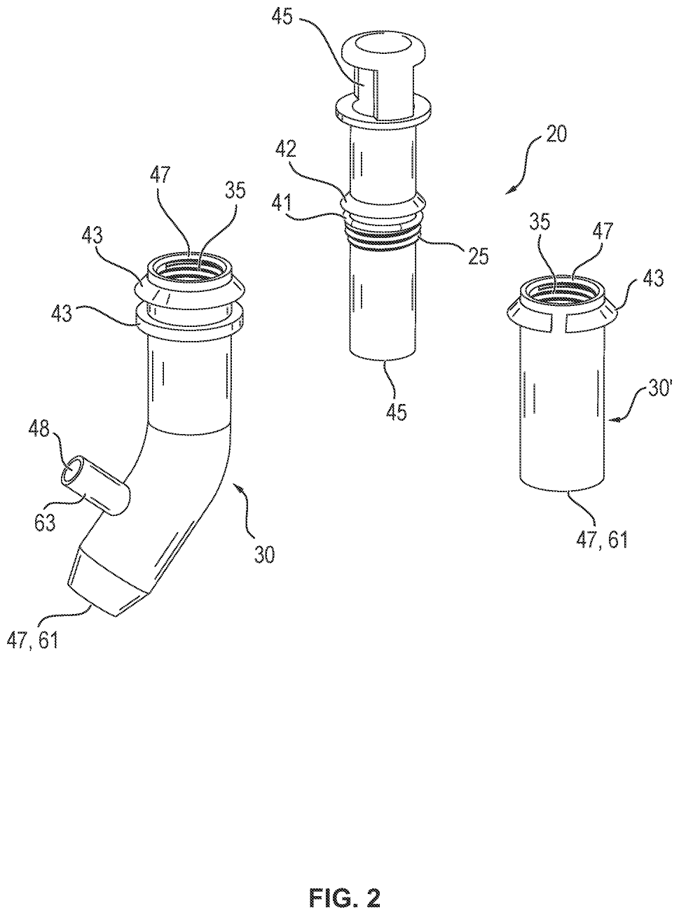

The two-part probe 5 consists of a probe element 20 and a conduit element 30. The conduit element 30 has a feedthrough channel 31 and is coupled to the pneumatic interface 3 by means of connecting elements 43. The probe element 20 has a probe channel 21 and is coupled to the breathing system 1 by means of a connection element 42. Further sealing elements on probe element 20 and conduit element 30 are not shown in this figure for reasons of clarity of the drawings; sealing elements 41 are shown in more detail in FIGS. 2 to 7. In FIG. 1, further elements of the anesthesia device 500 or ventilation device 700, such as a sensor interface 4 for coupling sensors, for example pressure sensors 300, temperature sensors 310, gas sensors 320, humidity sensors 330, flow rate sensors 340 with associated connections 27 are schematically also shown. The probe element 20 has at least one opening 45 to or into the breathing system 1, the conduit element 30 has at least one connection element 61 with openings 47, 48 to a connection 27 to the pneumatic interface 3, to valve arrangements 370, such as to actively controlled or passive valves, or to the sensor interface 4. The connections 27 enable gas exchange 29 between the breathing system 1 on the one hand and the sensor interface 4, the sensors 300, 310, 320, 330, 340 and valve arrangements 370 on the other hand.

In FIG. 2, a probe element 20, a conduit element 30, and another conduit element 30′ are shown in a perspective view. The probe element 20 can also be referred to as an upper part of the probe 5, the conduit element can also be referred to as a lower part of the probe 5. For the probe 5 itself, designations such as “grommet”, “socket”, “sleeve”, “socket”, “nozzle”, “grommet”, and “sleeve” are often used and are common. Identical elements in FIGS. 1 and 2 are designated with the same reference numerals as in FIG. 1. An opening 45 is arranged in the upper part of the probe element 20, which is provided for realizing a pneumatic connection with the breathing system 1 (FIG. 1). In addition, a sealing element 42 is provided which, together with a sealing element 41—in the form of a sealing ring (O-ring)—enables a gas-tight connection with the conduit elements 30, 30′. Arranged on the probe element 20 is an external thread element 25, which is configured and provided for assembly with an internal thread element 35 of the conduit element 30, 30′ with regard to the thread type, the thread pitch and the number of thread turns. Connecting elements 61, 63 with openings 47, 48 are arranged on the conduit elements 30, 30′, which enable a non-pneumatic connection, for example by means of hose lines, to a sensor interface 4 (FIG. 1). To arrange the conduit elements 30, 30′ on the pneumatic interface 3 (FIG. 1), sealing elements 43 are arranged on the conduit elements 30, 30′.

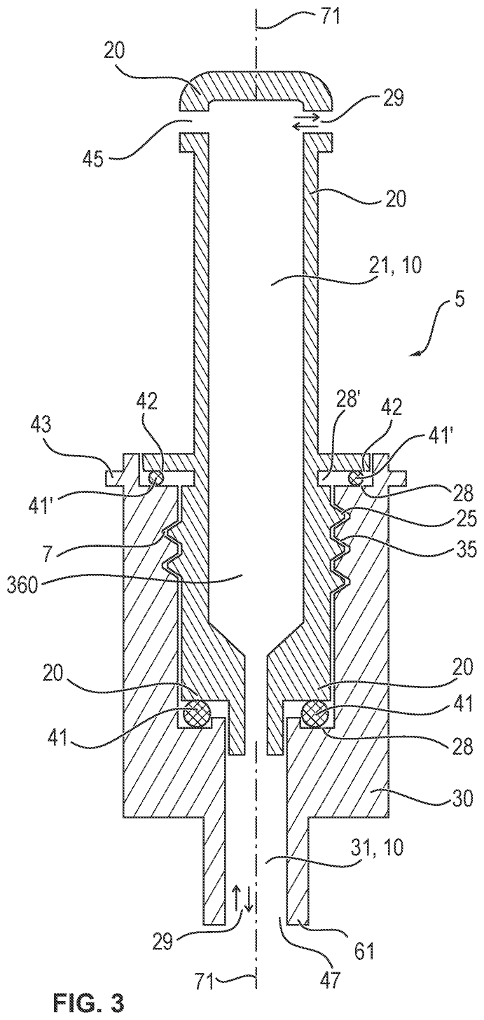

FIG. 3 shows a sectional view of a two-part probe according to FIG. 1. Identical elements in FIGS. 1, 2 and 3 are designated with the same reference numbers as in FIGS. 1 and 2. The probe 5 with probe element 20 with probe channel 21 and conduit element 30 with feed-through channel 31 is configured in a round shape symmetrically to a center line 71 with a connection element 61 and a probe opening 45. Sealing elements 41, 41′ are arranged in receptacles 28, 28′ in such a way that when a thread combination 7 is joined with screwing the probe element 20 by means of an externally threaded element 25 into the internally threaded element 35 of conduit element 30, a channel arrangement 10 is formed as a gas-tight connection for a gas exchange between the breathing system 1 (FIG. 1) and the pneumatic interface 3 (FIG. 1). The sealing with two sealing elements 41, 41′ at the two installation positions shown enables a sealing of a volume 360 located within the thread combination 7, 25, 35 between probe element 20 and conduit element 30 both against a gas inflow from the breathing system 1 (FIG. 1) and against a gas inflow from the direction of the pneumatic interface 3 (FIG. 1). The channel arrangement 10 thus forms, as it were, a volume element 360 with a closed volume 360.

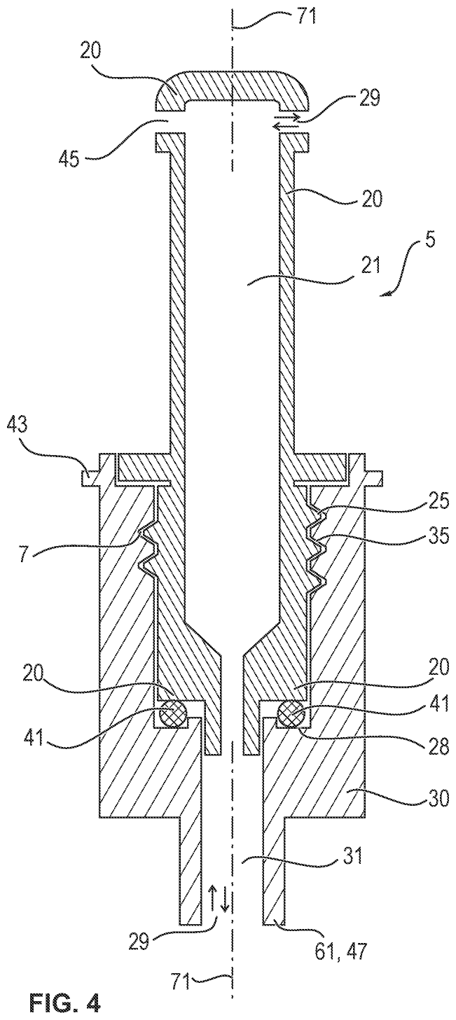

FIG. 4 shows an alternative simplified sealed variant of a two-part probe according to FIG. 3 in a sectional view. Identical elements in FIGS. 1, 2 , 3 and in FIG. 4 are designated with the same reference numerals as in FIGS. 1 to 3. The probe 5 with probe element 20 with probe channel 21 and conduit element 30 with feed-through channel 31 is configured in a round shape symmetrical to a center line 71 with a connection element 61 and a probe opening 45. A sealing element 41 is arranged in a receptacle 28 in such a way that when a thread combination 7 is joined with a screwing of the probe element 20 by means of an external thread element 25 into the internal thread element 35 of conduit element 30, a channel arrangement 10 is formed as a gas-tight connection for a gas exchange between the breathing system 1 (FIG. 1) and the pneumatic interface 3 (FIG. 1). The sealing element 41′ (FIG. 3) is not present in FIG. 4, the sealing between the probe element 20 and the conduit element 30 (FIG. 1) is effected exclusively by means of the one sealing element 41 and thereby also against a gas inflow from the direction of the pneumatic interface 3.

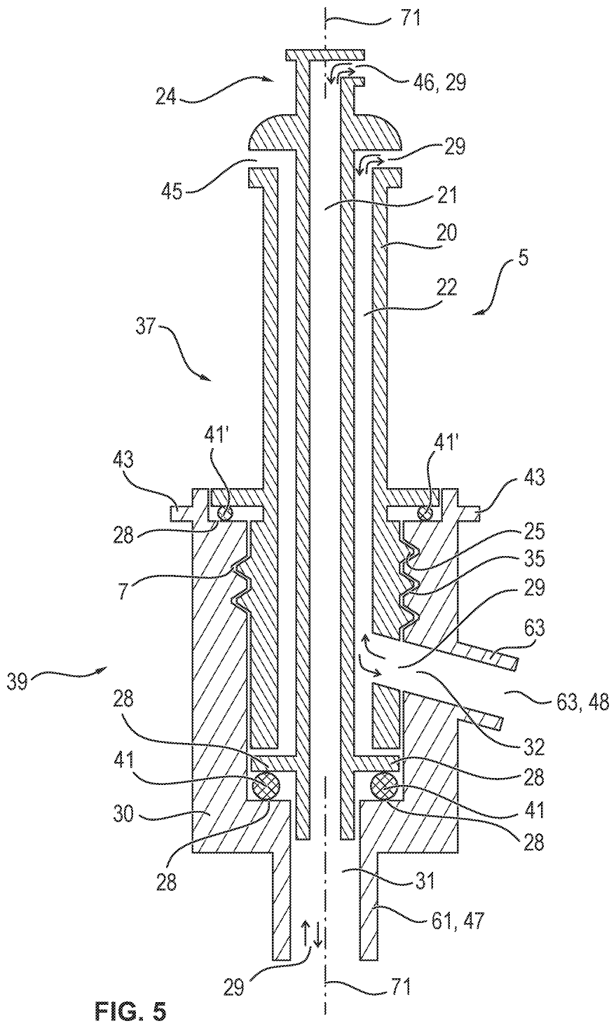

FIG. 5 shows a sectional view of a two-lumen, two-part probe. Identical elements in FIGS. 1 to 4 and in FIG. 5 are designated with the same reference numerals as in FIGS. 1 to 4. Two probe channels 21, 22 and two feed-through channels 31, 32 are arranged in both probe element 20 and conduit element 30. The first probe channel 21 and the first feed-through channel 31 are each arranged internally, inside a second probe channel 22, and a second feed-through channel 32, respectively, so that coaxial channel arrangements 37, 39 are formed in each case. The conduit element 31 is formed with two connection elements 61, 63, via whose openings 47, 48, for example, connections for gas exchange 29 with the sensor interface 4 (FIG. 1) and/or valve arrangements 370 (FIG. 1) are made possible. The probe element 20 has two openings 45, 46 to allow gas exchange 29 to the breathing system 1 (FIG. 1). In this FIG. 5, the openings in a probe head 24 are shown at different heights, in this way, for example, an addition by means of one of the two openings 45, 46 and a gas continuation or pressure measurement by means of the other of the two openings 45, 46 can be largely decoupled from each other. This can be advantageous in operation with gas measurement and gas dosing for an anesthesia device or ventilator device. In other embodiments of the probe element 20, or of the probe head 23, the openings may be configured in a different way, for example at the same height, if this is advantageous for operation with anesthesia or ventilation equipment. The further construction of the probe 5 according to this FIG. 5 corresponds essentially to the construction of the probe 5 according to FIGS. 3 and 4, whereby the aspects and functions of sealing and gas exchange 39 mentioned for FIGS. 3 and 4 are thus also transferable to this embodiment according to FIG. 5.

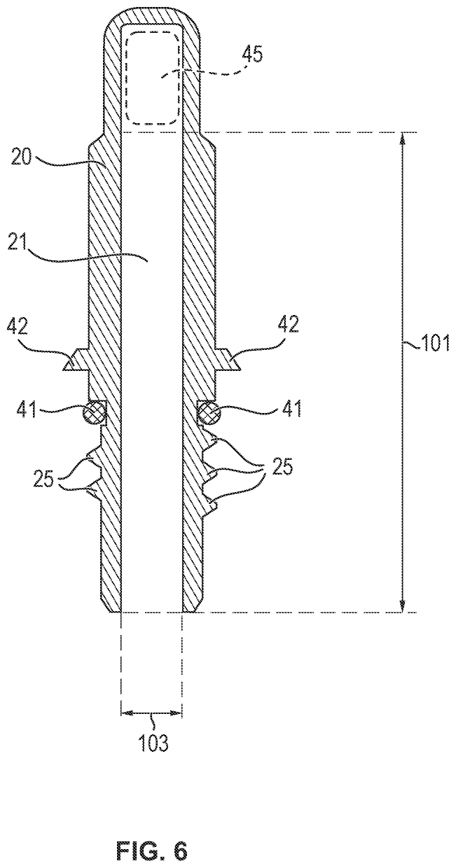

In FIG. 6, the dimensions of a probe element 20 with a probe channel 21 with a length 101 and a diameter 103 as components of a two-part probe 5 according to FIG. 2 are shown as an example. The external thread 25, an opening 45 in the probe element 20, a sealing element 42 as a mechanical stop for coupling the probe element 20 to a conduit element 30 according to FIG. 2 and a sealing element 41 formed as an O-ring are schematically indicated. Identical elements in FIGS. 1 to 5 and FIG. 6 are designated with the same reference numerals as in FIGS. 1 to 5. The dimensioning of the length 100 and the diameter 103 results from the dimensioning rule according to the formula 3 derived and explained in the description above, taking into account the assumptions and ambient conditions on which this is based. This results in a length 101 in a range of 45.70 mm to 46.30 mm and a diameter 103 in a range of 5.75 mm to 6.25 mm for the probe channel 21.

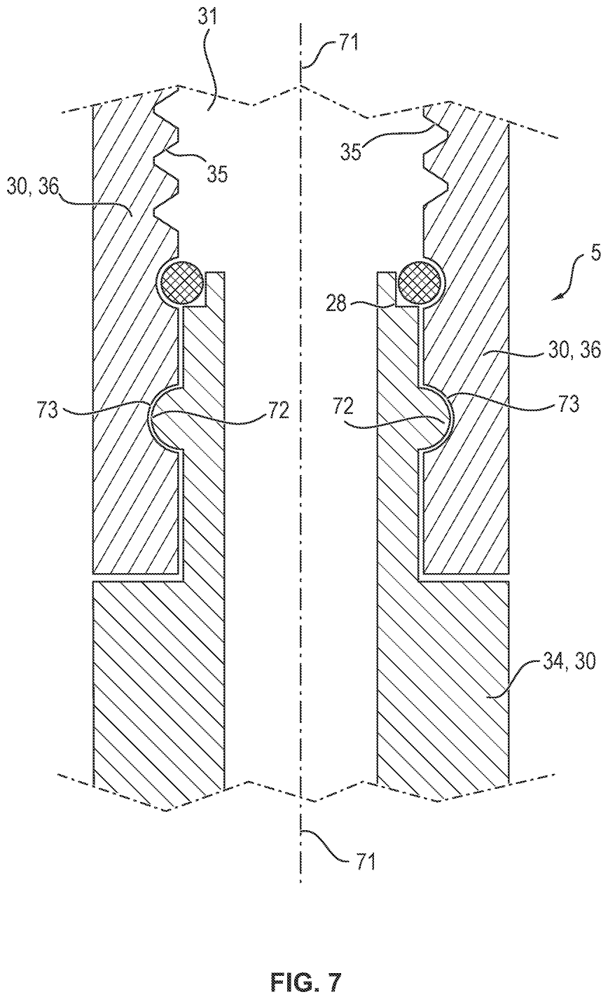

FIG. 7 shows a sectional view of a probe 5 with a connecting element 30. Identical elements in FIGS. 1 to 6 and in FIG. 7 are designated with the same reference numerals as in FIGS. 1 to 6. The conduit element 30 is formed in two parts from an upper part 36 and a rotatable lower part 34. The further structure of the probe 5 according to this FIG. 7 corresponds essentially to the structure of the probe 5 according to FIGS. 3 and 4, whereby the aspects and functions of sealing and gas exchange 39 mentioned for FIGS. 3 and 4 are thus also transferable to this embodiment according to FIG. 7. The lower part 34, which is rotatable in relation to the upper part 36, enables a simplified connection of tube connections to the pneumatic interface 3 (FIG. 1) in an anesthesia device or ventilator device. The rotatability is made possible by two corresponding elements 72, 73 in a tongue and groove type connection. The further structure according to this FIG. 7 corresponds essentially to the structure of the probe 5 according to FIGS. 3 and 4, whereby the aspects mentioned for FIGS. 3 and 4 are thus also transferable to this embodiment according to FIG. 7.

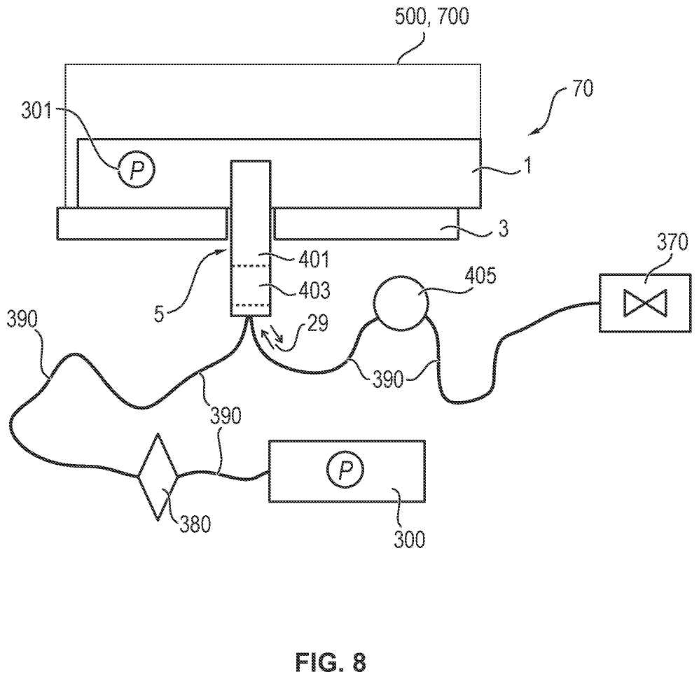

In FIG. 8 shows a schematic representation of a pneumatic situation 70 in and on the breathing system 1 and the pneumatic interface 3 according to FIG. 1. This FIG. 8 is used for understanding the derivations of the formulas 1 to 3 for configuring the buffer volume of the probe 5 in the description above. Identical elements in FIGS. 1 to 7 and in FIG. 8 are designated with the same reference numerals as in FIGS. 1 to 7. The pressure in the breathing system 301 (p_Atemsystem) is supplied to the pressure sensor 300 via the probe 5 by means of the pneumatic interface 3 in the gas exchange 29 and a hose line 390 (V_Hose) via a hygiene filter 380 (V_Filter). Gas volumes are fed from the valve assembly 370 via hose lines 390 (V_hose) and possibly also other components 405 (V_div) into the breathing system 1 by means of the pneumatic interface 3 in the gas exchange 29 via the probe 5. The probe 5 has a volume 401 (V_probe). Pressure increases in the breathing system 1 result in a compressed volume 403 (dV_compression) in the probe 5.

With the principal arrangement of components 1, 3, 5, 300, 370, 380, 390, 401, 403, 405 as shown in FIG. 8, Formulas 1 to 3 in the general description can be understood and implemented.

While specific embodiments of the invention have been shown and described in detail to illustrate the application of the principles of the invention, it will be understood that the invention may be embodied otherwise without departing from such principles.

### REFERENCE DIGIT LIST

1 Breathing system
3 Pneumatic interface
4 Sensor interface with sensors
5 Two-piece configured probe (two part probe)
7 Thread combination
10 Channel arrangement
20 Probe element
21 First probe channel
22 Second probe channel
23 Probe head
25 Male threaded element
27 Connection, screw connection
28 Mounting for sealing element, heel
29 Gas exchange
30, 30′ Lead-through element
31 First flowthrough (conduit) channel
32 Second flowthrough (conduit) channel
34 Rotatable lower part of the conduit element
35 Internally threaded element
36 Upper part of the conduit element
37, 39 Coaxial channel arrangements
41, 41′, 41″ Sealing elements
42, 43 Sealing element
45, 46 Probe openings, openings in the probe element
47, 48 Openings in the conduit element
61, 63 Connection elements
70 Physical situation
71 Centerline
72, 73 Corresponding elements
100 Arrangement
101 Length
103 Diameter
200 Pneumatic gas supply system with arrangement 100
300 Pressure sensor
301 Pressure in the breathing system
310 Temperature sensor
320 Gas sensor
330 Humidity sensor
340 Flow rate sensor
360 Volume element, volume
370 Actuators, valve assembly
380 Filter
390 Hose lines, hose volume
401 Volume of the probe (V_probe)
403 Compressed volume (dV_compression)
405 Other volume (V_div)
500 Anesthesia machine
700 Ventilator

What is claimed is:

1. A probe for a pneumatic gas delivery system with a breathing system and a pneumatic interface of a ventilator or anesthesia machine, the probe comprising:
a probe element;
a conduit element, wherein the probe is a combination of the probe element with the conduit element as a two part configuration with the combination of the probe element and the conduit element forming a probe channel and a feedthrough channel of at least one channel arrangement internal to the probe to provide a pneumatic or fluidic conductive connection between the breathing system and the pneumatic interface, wherein the probe element is connectable to the conduit element and the probe element together with the conduit element forms the probe in an interconnected state, wherein a fluid exchange connection between the probe element and the conduit element is enabled by the channel arrangement; and
a connection element arranged on the conduit element, wherein the probe is connectable to the pneumatic interface or to components of the ventilator or to components of the anesthesia machine by the connection element.

2. A probe according to claim 1, wherein the probe is configured as a measuring probe for a gas exchange via the pneumatic interface and comprises at least one probe opening arranged in the probe element, wherein via the at least one probe opening, the probe channel inside the probe element and the feedthrough channel inside the conduit element, a transmission of a pneumatic flow rate prevailing in the breathing system is enabled to transmit a pneumatic and/or physical situation prevailing in the breathing system to a sensor interface connected to a pneumatic interface or to sensors connected to the pneumatic interface.

3. A probe according to claim 1, wherein the probe is configured as a gas inlet for a gas exchange by the probe and comprises at least one probe opening arranged in the probe element, wherein via the at least one probe opening, the probe channel inside the probe element and the feedthrough channel inside the conduit element, a flow of gas quantities is enabled from the pneumatic interface into the breathing system.

4. A probe according to claim 1, wherein the probe is configured as a gas outlet for a gas exchange by the probe and comprises at least one probe opening arranged in the probe element, wherein via the at least one probe opening, the probe channel inside the probe element and the feedthrough channel inside the conduit element, a flow of gas quantities is enabled from the breathing system into the pneumatic interface.

5. A probe according to claim 1, wherein the fluid exchange connection between the probe element and the conduit element is formed as a releasable screw connection as a threaded combination with an externally threaded element and an internally threaded element corresponding thereto.

6. A probe according to claim 1, wherein the fluid exchange connection between the probe element and the conduit element is formed as a tool-less, releasable connection.

7. A probe according to claim 1, wherein the probe element further comprises a second probe channel and the conduit element further comprises a second feedthrough channel.

8. A probe according to claim 7, wherein the probe element comprises a probe opening and further comprises a further probe opening to provide a pneumatic or fluidic conductive connection between the breathing system and the pneumatic interface via the second probe channel and the second feedthrough channel, wherein the second probe channel and the probe channel form a coaxial arrangement with each other, wherein one of the probe channels is internally disposed in the other probe channel, and wherein the second feedthrough channel and the feedthrough channel form a coaxial arrangement with each other and wherein one of the feedthrough channels is internally disposed in the other feedthrough channel.

9. A probe according to claim 8, wherein a volume element is arranged or integrated in or on the probe, the probe element, the conduit element, the channel arrangement, one or more of the probe channels, one or more of the feedthrough channels or the connection element, and wherein the volume element is configured to buffer pressure differences, reduce pressure surges in the breathing system or the pneumatic interface or dampen movement of gas quantities between the breathing system and the pneumatic interface.

10. A probe according to claim 1, wherein a volume element is arranged or integrated in or on the probe, the probe element, the conduit element, the channel arrangement, the probe channel, the feedthrough channel or the connection element and wherein the volume element is configured to buffer pressure differences, reduce pressure surges in the breathing system or the pneumatic interface or dampen movement of gas quantities between the breathing system and the pneumatic interface.

11. A probe according to claim 1, wherein the probe element and/or the conduit element comprise at least one sealing element providing a gas-tight connection between the probe element and the conduit element, and wherein the conduit element comprises at least one sealing element providing a gas-tight between the conduit element and the pneumatic interface.

12. A probe according to claim 11, wherein:
the probe and/or the conduit element and/or the probe element and/or at least one of the sealing elements is formed of a plastic material, and/or
the probe and/or the conduit element and/or the probe element is formed of a plastic material or a metallic material.

13. A probe according to claim 1, in combination with the breathing system and the pneumatic interface to form an arrangement, the probe being arranged on the breathing system, wherein:
with the probe configured as a measuring probe, a detection of a pneumatic and/or physical situation in the breathing system is enabled, and/or
with the probe configured as a gas inlet, a flow of gas quantities is enabled from the pneumatic interface into the breathing system; and/or
with the probe configured as a gas outlet, a flow of gas quantities is enabled from the breathing system into the pneumatic interface.

14. A probe according to claim 13, wherein the arrangement comprises a pneumatic gas delivery system or an anesthesia machine or a ventilator and further comprises:
at least one pressure sensor; and/or
at least one temperature sensor; and/or
at least one gas sensor; and/or at least one humidity sensor; and/or
at least one flow rate sensor.

15. A probe according to claim 13, wherein the arrangement further comprises at least one connection line between the connection element at the conduit element of the probe with the pressure sensor and/or the temperature sensor and/or the gas sensor and/or the humidity sensor and/or the flow rate sensor.

16. A probe according to claim 13, wherein the arrangement further comprises a second probe to provide at least two probes, wherein pneumatic or fluidic connections between the breathing system and the interface via the two probes are formed by mutually unique and unmistakable configurations of a thread combination on the two probe elements and/or unique and unmistakable configurations of the connection elements on the conduit element of the two probe elements.

17. A gas delivery system comprising:
a breathing system;
a pneumatic interface; and
a probe comprising: a probe element; a conduit element, wherein the probe is a combination of the probe element with the conduit element as a two part configuration with the combination of the probe element and the conduit element forming a probe channel and a feedthrough channel of at least one channel arrangement internal to the probe to provide a pneumatic or fluidic conductive connection between the breathing system and the pneumatic interface, wherein the probe element is connectable to the conduit element and the probe element together with the conduit element forms the probe in an interconnected state, wherein a fluid exchange connection between the probe element and the conduit element is enabled by the channel arrangement; and a connection element arranged on the conduit element, wherein the probe is connectable to the pneumatic interface or to components of the ventilator or to components of the anesthesia machine by the connection element, the probe being arranged on the breathing system, wherein:
with the probe configured as a measuring probe, a detection of a pneumatic and/or physical situation in the breathing system is enabled, and/or
with the probe configured as a gas inlet, a flow of gas quantities is enabled from the pneumatic interface into the breathing system; and/or
with the probe configured as a gas outlet, a flow of gas quantities is enabled from the breathing system into the pneumatic interface.

18. A gas delivery system according to claim 17, wherein the gas delivery system comprises a pneumatic gas delivery system or an anesthesia machine or a ventilator and further comprises:
at least one pressure sensor; and/or
at least one temperature sensor; and/or
at least one gas sensor; and/or at least one humidity sensor; and/or
at least one flow rate sensor.

19. A gas delivery system according to claim 18, further comprising at least one connection line between the connection element at the conduit element of the probe with the pressure sensor and/or the temperature sensor and/or the gas sensor and/or the humidity sensor and/or the flow rate sensor.

20. A gas delivery system according to claim 18, further comprising a second probe to provide at least two probes, wherein pneumatic or fluidic connections between the breathing system and the interface via the two probes are formed by mutually unique and unmistakable configurations of a thread combination on the two probe elements and/or unique and unmistakable configurations of the connection elements on the conduit element of the two probe elements.
